(19) United States
(12) Patent Application Publication
(10) Pub. No.: US 2023/0046598 A1
(43) Pub. Date: Feb. 16, 2023

(54) MACHINE LEARNING SYSTEM FOR INTERPRETING HOST PHAGE RESPONSE

(71) Applicant: Adaptive Phage Therapeutics, Inc., Gaithersburg, MD (US)

(72) Inventors: Rob Cohen, Kensington, MD (US); Greg Merril, Bethesda, MD (US); Miguel Barreto-Sanz, Lausanne (CH); Priyesh Prateek Agrawal, Prayagraj (IN)

(73) Assignee: Adaptive Phage Therapeutics, Inc., Gaithersburg, MD (US)

(21) Appl. No.: 17/789,862

(22) PCT Filed: Dec. 23, 2020
(86) PCT No.: PCT/US20/66788

§ 371 Date: Jun. 29, 2022

Related U.S. Application Data
(60) Provisional application No. 62/955,995, filed on Dec. 31, 2019.

(51) Int. Cl. G06N 20/00

(52) U.S. Cl. CPC G06N 20/00

(57) ABSTRACT

A computer implemented method of generating a machine learning model for interpreting host phage response data comprising receiving datasets and labels for a host phage response, training a machine learning model and using this model to estimate the efficacy of a test phage in inhibiting growth of a test bacteria.

### TECHNICAL FIELD

The present disclosure relates to Machine Learning based methods for interpreting host-phage response data.

### BACKGROUND

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Multiple drug resistant (MDR) bacteria are emerging at an alarming rate. Currently, it is estimated that at least 2 million infections are caused by MDR organisms every year in the United States leading to approximately 23,000 deaths. Moreover, it is believed that genetic engineering and synthetic biology may also lead to the generation of additional highly virulent microorganisms.

For example, Staphylococcus aureus are gram positive bacteria that can cause skin and soft tissue infections (SSTI), pneumonia, necrotizing fasciitis, and blood stream infections. Methicillin resistant S. aureus (“MRSA”) is an MDR organism of great concern in the clinical setting as MRSA is responsible for over 80,000 invasive infections, close to 12,000 related deaths, and is the primary cause of hospital acquired infections. Additionally, the World Health Organization (WHO) has identified MRSA as organisms of international concern.

In view of the potential threat of rapidly occurring and spreading virulent microorganisms and antimicrobial resistance, alternative clinical treatments against bacterial infection are being developed. One such potential treatment for MDR infections involves the use of phage. Bacteriophages (“phages”) are a diverse set of viruses that replicate within and can kill specific bacterial hosts. The possibility of harnessing phages as an antibacterial was investigated following their initial isolation early in the 20th century, and they have been used clinically as antibacterial agents in some countries with some success. Notwithstanding, phage therapy was largely abandoned in the U.S. after the discovery of penicillin, and only recently has interest in phage therapeutics been renewed.

The successful therapeutic use of phage depends on the ability to administer a phage strain that can kill or inhibit the growth of a bacterial isolate associated with an infection. Empirical laboratory techniques have been developed to screen for phage susceptibility on bacterial strains (i.e. efficacy at inhibiting bacterial growth). However, these techniques are time consuming and subjective, and involve attempting to grow a bacterial strain in the presence of a test phage. After many hours an assessment of the capability of the phage to lyse (kill) or inhibit bacterial growth is estimated (the host-phage response) by manual, visual inspection.

One such test is the plaque assay which is a semi-solid medium assay which measures the formation of a clear zone in bacterial lawn resulting from placement of a test phage and infection of the bacteria. Although the plaque assay is simple, plaque morphologies and sizes can vary with the experimenter, media and other conditions. More recently an automated high throughput, indirect liquid lysis assay system has been developed to evaluate phage growth using the OmniLog™ system (Biolog, Inc). The OmniLog™ system is an automated plate-based incubator system coupled to a camera and computer which, using redox chemistry, employs cell respiration as a universal reporter. The wells in the plate each contain growth medium, a tetrazolium dye, a (host) bacterial strain and a phage (along with control/calibration wells). During active growth of bacteria, cellular respiration reduces a tetrazolium dye and produces a color change. Successful phage infection and subsequent growth of the phage in its host bacterium results in reduced bacterial growth and respiration and a concomitant reduction in color. The camera collects images at a plurality of time points, and each well in an image is analysed to generate a color measure. This can be referenced to the initial color, or a reference color, so that a time series dataset of colour change over time is collected (i.e. a colorimetric assay). The time series dataset for each well (i.e. host-phage combination) is graphed, and a user then (subjectively) reviews each of the graphs (e.g. 96 graphs for a 96 well plate). The user uses his/her experience, intuitive and implicit knowledge to interpret the graph and estimate the host-phage response. This leads to increased variability or quality as the interpretation is subjective and dependent on the skill level and/or the attentiveness of the user reviewing the graphs on a particular day.

Thus, there is a need to develop improved automated methods for analysing/interpreting host phage response data, for example to reduce variability based on a human's interpretation, or to at least provide a useful alternative to existing methods. Further, an automated approach will reduce variability based on a human's interpretation.

### SUMMARY

According to a first aspect, there is provided a computer implemented method for training a machine learning model for interpreting host phage response data, the method comprising:

receiving or uploading, by a computing system, a host phage response dataset and labels, wherein the host phage response dataset comprises a time series dataset for each of a plurality of host-phage combinations in which a host bacteria is grown in the presence of a phage, and each data point in the time series dataset associated with a host-phage combination comprises a measurement of a parameter indicative of the growth of the respective host bacteria in the presence of the respective phage at a specific time, and each time series dataset has an associated label indicating an efficacy of the phage in inhibiting growth of the host bacteria;

fitting for each time series dataset, at least one function over a first time window;

generating a set of summary parameters for each fit, the summary parameters comprising one or more model coefficients, goodness of fit, R², errors, residuals, or summary statistics of residuals; and

training a machine learning model on a training dataset comprising the set of summary parameters for each fit to one of the time series datasets, and the associated label for the fitted time series dataset;

exporting or saving the machine learning model in an electronic format for subsequent use to estimate an efficacy of a test phage in inhibiting growth of a test bacteria using a host phage response time series dataset obtained using the test phage and test bacteria.

In one form, fitting, for each time series data set, at least one function over a first time window comprises fitting a single function over the first time window. In another form, fitting, for each time series data set, at least one function over a first time window comprises fitting at least two functions over the first time window, wherein each of the functions have a different functional form. In another form fitting, for each time series data set, at least one function over a first time window comprises performing a plurality of fits, wherein each fit comprises fitting a function over a time segment wherein the first time window is defined by a start of the earliest time segment and the end of a latest time segment and each time segment is shorter than the first time window. The time segments may be contiguous or non-contiguous time segments. In one form a number of time segments is at least three. In one form the end of the first time period is 24 hours or less. In one form the at least one function one or more of a linear function or a polynomial functional.

In one form the machine learning model is a binary classifier which generates a binary outcome indicating whether a test phage is efficacious in inhibiting growth of a test bacteria or not. In another form the machine learning model is a probabilistic classifier which estimates a probability that a test phage is efficacious in inhibiting growth of a test bacteria.

According to a second aspect, there is provided a computer implemented method for interpreting host phage response data, the method comprising:

loading, by a computer system a trained machine learning model stored in an electronic format and configured to classify host response dataset;

receiving and/or uploading a host response dataset for a test phage, wherein the host response dataset comprises a time series dataset where each data point in the time series dataset comprises a measurement of a parameter indicative of the growth of a host bacteria in the presence of the test phage at a specific time

fitting at least one function over a first time window;

generating a set of summary parameters for the fitting;

obtaining an estimate of an efficacy of the test phage in inhibiting growth of the host bacteria by providing the set of summary parameters to the trained machine learning model;

reporting the estimate of the efficacy of the test phage.

In one form the method may further comprise receiving an updated host response dataset comprising additional data points and repeating the fitting, generating, obtaining and reporting steps, wherein reporting the estimate includes an estimate of the probability that the test phage is efficacious.

In one form, the method may be repeated for a plurality of host response datasets, and the method further comprises:

obtaining a set of at least two test phage estimated as efficacious against a test bacteria;

obtaining estimates of one or more mechanisms of action for each test phage in the set;

obtaining a measure of diversity for each pair of test phage in the set based on the estimated mechanisms of action for each test phage;

selecting at least two phage for use in a therapeutic phage formulation based on the obtained measures of diversity.

In preferred embodiments the mechanism of action for each test phage is determined by sequencing the test phage.

The above methods may be implemented in a non-transitory, computer program product comprising instructions to implement any of the above methods in a computing apparatus. The above methods may also be implemented in a computing apparatus comprising at least one memory and at least one processor configured to implement the above methods.

According to a third aspect, there is provided a non-transitory, computer program product comprising computer executable instructions for training a machine learning model for interpreting host phage response data, the instructions comprising:

receive a host phage response dataset and labels, wherein the host phage response dataset comprises a time series dataset for each of a plurality of host-phage combinations in which a host bacteria is grown in the presence of a phage, and each data point in the time series dataset associated with a host-phage combination comprises a measurement of a parameter indicative of the growth of the respective bacteria in the presence of the respective phage at a specific time, and each time series dataset has an associated label indicating the efficacy of the phage in inhibiting growth of the host bacteria;

fit, for each time series data set, at least one function over a first time window;

generate a set of summary parameters for each fit, the summary parameters comprising one or more model coefficients, goodness of fit, R², errors, residuals, or summary statistics of residuals; and

train a machine learning model on a training dataset comprising the set of summary parameters for each fit to one of the time series dataset, and the associated label for the time series dataset;

export the machine learning model in an electronic format.

According to a fourth aspect, there is provided a non-transitory, computer program product comprising computer executable instructions for interpreting host phage response data, the instructions executable by a computer to:

load a trained machine learning model configured to classify host response dataset;

receive a host response dataset for a test phage, wherein the host response dataset comprises a time series dataset where each data point in the time series dataset comprises a measurement of a parameter indicative of the growth of a host bacteria in the presence of the test phage at a specific time

fit at least one function over a first time window;

generate a set of summary parameters for the fitting;

obtain an estimate of an efficacy of the test phage in inhibiting growth of the host bacteria by providing the set of summary parameters to the trained machine learning model;

report the estimate of the efficacy of the test phage.

According to a fifth aspect, there is provided a computing apparatus comprising:

at least one memory, and

at least one processor wherein the memory comprises instructions to configure the processor to:

receive a host phage response dataset and labels, wherein the host phage dataset comprises a time series dataset for each of a plurality of host-phage combinations in which a host bacteria is grown in the presence of a phage, and each data point in the time series dataset associated with a host-phage combination comprises a measurement of a parameter indicative of the growth of the respective bacteria in the presence of the respective phage at a specific time, and each time series dataset has an associated label indicating the efficacy of the phage in inhibiting growth of the host bacteria;

fit, for each time series data set, at least one function over a first time window

generate a set of summary parameters for each fit, the summary parameters comprising one or more model coefficients, goodness of fit, R², errors, residuals, or summary statistics of residuals; and

train a machine learning model on a training dataset comprising the set of summary parameters for each fit to one of the time series datasets, and the associated label for the fitted time series dataset;

export or save the machine learning model in an electronic format, wherein in use, the trained machine learning model is used to estimate the efficacy of a test phage in inhibiting growth of a test bacteria using a host phage response time series dataset obtained using the test phage and test bacteria.

According to a sixth aspect, there is provided a computing apparatus comprising:

at least one memory, and

at least one processor wherein the memory comprises instructions to configure the processor to:

load a trained machine learning model configured to classify a host response dataset;

receive a host response dataset for a test phage, wherein the host response dataset comprises a time series dataset where each data point in the time series dataset comprises a measurement of a parameter indicative of the growth of a host bacteria in the presence of the test phage at a specific time

fit at least one function over a first time window;

generate a set of summary parameters for the fitting;

obtain an estimate of an efficacy of the test phage in inhibiting growth of the host bacteria by providing the set of summary parameters to the trained machine learning model;

report the estimate of the efficacy of the test phage.

According to a sixth aspect, there is provided a therapeutic phage formulation comprising at least two phage, wherein the at least two phage were selected by:

obtaining a set of at least two test phage estimated as efficacious against a test bacteria through using a trained machine learning model configured to interpret host phage response data for a plurality of host-phage combinations in which a host bacteria is grown in the presence of a phage;

obtaining estimates of one or more mechanisms of action for each test phage in the set;

obtaining a measure of diversity for each pair of test phage in the set based on the estimated mechanisms of action for each test phage;

selecting at least two phage for use in the therapeutic phage formulation based on the obtained measures of diversity.

In preferred embodiments the mechanism of action for each test phage is determined by sequencing the test phage.

### BRIEF DESCRIPTION OF DRAWINGS

Embodiments of the present disclosure will be discussed with reference to the accompanying drawings wherein:

FIG. 1 is a flow chart of a method for training a machine learning model for interpreting host phage response data according to an embodiment

FIG. 2 is a plot of a plurality of host-phage response datasets according to an embodiment;

FIG. 3 is a schematic diagram of a computing apparatus according to an embodiment;

FIG. 4 is a comparison of several curve fits on a first host-phage time series dataset where the phage does not inhibit growth of the bacterial host (not efficacious/ineffective), and a second host-phage time series dataset where the phage does inhibit growth of the bacterial host (efficacious/effective) according to an embodiment; and

FIG. 5 is a set of plots showing the time taken for a machine learning model to correctly classify the efficacy of a host-phage time series dataset according to an embodiment;

In the following description, like reference characters designate like or corresponding parts throughout the figures.

### DESCRIPTION OF EMBODIMENTS

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules. “A phage formulation” can mean at least one phage formulation, as well as a plurality of phage formulations, i.e., more than one phage formulation. As understood by one of skill in the art, the term “phage” can be used to refer to a single phage or more than one phage.

The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, a “subject” is a vertebrate, preferably a mammal, more preferably a human. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed. Preferably, a “subject” encompasses any organisms, e.g., any animal or human, that may be suffering from a bacterial infection, particularly an infection caused by a multiple drug resistant bacterium.

As understood herein, a “subject in need thereof” includes any human or animal suffering from a bacterial infection, including but not limited to a multiple drug resistant bacterial infection, a microbial infection or a polymicrobial infection. Indeed, while it is contemplated herein that the methods may be used to target a specific pathogenic species, the method can also be used against essentially all human and/or animal bacterial pathogens, including but not limited to multiple drug resistant bacterial pathogens. Thus, in a particular embodiment, by employing the methods of the present invention, one of skill in the art can design and create personalized phage formulations against many different clinically relevant bacterial pathogens, including multiple drug resistant (MDR) bacterial pathogens.

As understood herein, an “effective amount” of a pharmaceutical composition refers to an amount of the composition suitable to elicit a therapeutically beneficial response in the subject, e.g., eradicating a bacterial pathogen in the subject. Such response may include e.g., preventing, ameliorating, treating, inhibiting, and/or reducing one of more pathological conditions associated with a bacterial infection.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term “about” means within an acceptable error range for the particular value, such as ±1-20%, preferably ±1-10% and more preferably ±1-5%. In even further embodiments, “about” should be understood to mean +/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition is described as containing characteristics A, B, and/or C, the composition can contain A feature alone; B alone; C alone; A and B in combination; A and C in combination; B and C in combination; or A, B, and C in combination.

The term “phage sensitive” or “sensitivity profile” means a bacterial strain that is sensitive to infection and/or killing by phage and/or in growth inhibition. That is phage is efficacious or effective in inhibiting growth of the bacterial strain.

The term “phage insensitive” or “phage resistant” or “phage resistance” or “resistant profile” is understood to mean a bacterial strain that is insensitive, and preferably highly insensitive to infection and/or killing by phage and/or growth inhibition. That is phage is not efficacious or ineffective in inhibiting growth of the bacterial strain.

A “therapeutic phage formulation”, “therapeutically effective phage formulation”, “phage formulation” or like terms as used herein are understood to refer to a composition comprising one or more phage which can provide a clinically beneficial treatment for a bacterial infection when administered to a subject in need thereof.

As used herein, the term “composition” encompasses “phage formulations” as disclosed herein which include, but are not limited to, pharmaceutical compositions comprising one or more purified phage. “Pharmaceutical compositions” are familiar to one of skill in the art and typically comprise active pharmaceutical ingredients formulated in combination with inactive ingredients selected from a variety of conventional pharmaceutically acceptable excipients, carriers, buffers, and/or diluents. The term “pharmaceutically acceptable” is used to refer to a non-toxic material that is compatible with a biological system such as a cell, cell culture, tissue, or organism. Examples of pharmaceutically acceptable excipients, carriers, buffers, and/or diluents are familiar to one of skill in the art and can be found, e.g., in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water, saline, and glycerol.

As used herein, the term “estimating” encompasses a wide variety of actions. For example, “estimating” may include calculating, computing, processing, determining, deriving, investigating, looking up (e.g., looking up in a table, a database or another data structure), ascertaining and the like. Also, “estimating” may include receiving (e.g., receiving information), accessing (e.g., accessing data in a memory) and the like. Also, “estimating” may include resolving, selecting, choosing, establishing and the like.

Embodiments of computer implemented methods and systems for training a machine learning model for interpreting host phage response, and then subsequent use of the machine learning model for interpreting host phage response will now be described.

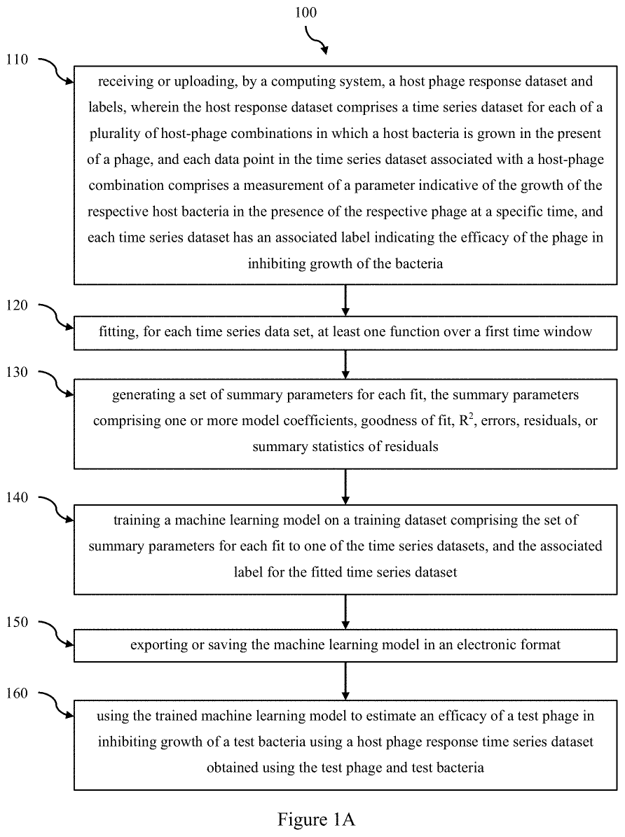

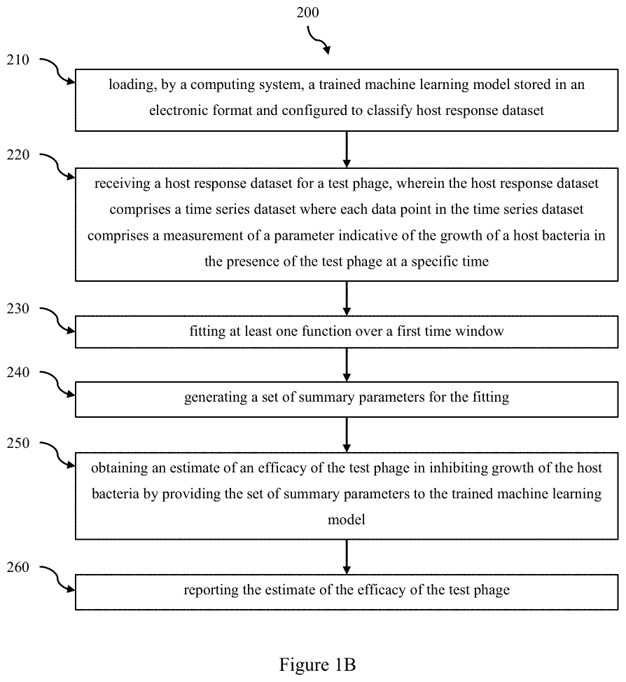

FIG. 1A is a flow chart of a method 100 for training a machine learning model for interpreting host phage response data according to an embodiment and FIG. 1B is flowchart of method 200 for interpreting host phage response data using the trained machine learning model.

Referring to FIG. 1A the method for training a machine learning model for interpreting host phage response data 100 comprises receiving a host phage response dataset and labels 110. The dataset comprises a time series dataset for each of a plurality of host-phage combinations. Each data point in the time series dataset associated with a host-phage combination comprises a measurement of a parameter indicative of the growth of the respective bacteria in the presence of the respective phage at a specific time. Further, for training purposes, each time series dataset has an associated label indicating the efficacy of the phage in inhibiting growth of the bacteria. To be clear, the indicator of growth includes indicating a lack of growth such as an indicator of lysis of the bacteria by the test phage. This will typically be a binary flag or value, such as “1” or “TRUE” for efficacious (i.e. inhibits or lyses bacteria) and “0” or “FALSE” for inefficacious/ineffective. In some embodiments the value could be a probability estimate, and a threshold value could be determined or varied to classify the time series dataset.

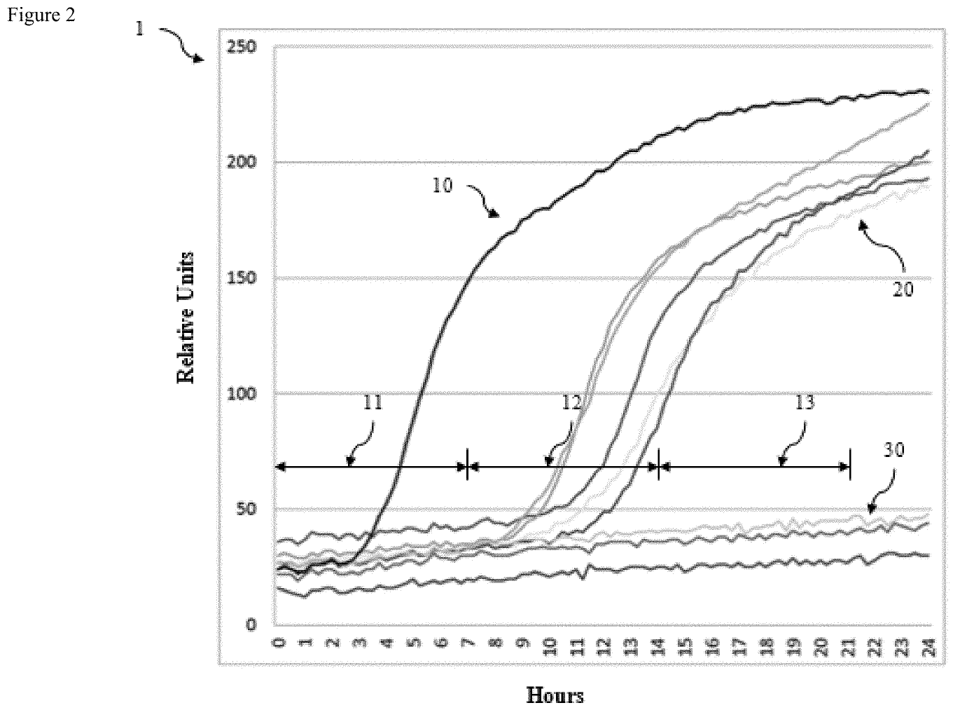

FIG. 2 is a plot 1 of a plurality of host-phage response datasets 10 according to an embodiment. These plot an indicator of growth in arbitrary units as a function of time. The indicator of growth may be measure of a dye colour or other indicator of bacterial growth or respiration, as well as measures lack of growth such as a measure of lysis of bacteria, including colorimetric and non-colorimetric measures. Over time, the datasets split into two groups of datasets. The first set 20 of host-phage responses correspond to responses in which the phage are non-efficacious—that the phage are ineffective and have a S shaped growth curve (i.e. sigmoidal) starting with an initial lag phase (or time period) 11, followed by growth phase (or time period) 12 in which the bacteria continues to grow in the presence of the phage, and a stabilisation phase (or time period) 13 in which growth of the bacteria stabilises, for example as it has fully colonised the well or reached some growth limit. In the second set 30 of host-phage responses correspond to responses in which the phage are efficacious—that the phage are effective in inhibiting growth of the bacteria and the growth curve is linear and fairly flat or slightly rising over time.

Rather than train the machine learning model directly on the images of each well (i.e. host-phage response), or the time series dataset of each well, one or more functions are first fitted to the time series dataset for each host-phage combination (i.e. each well) over a first time window. The first time window may be a subset of the time spanned by the time series dataset. For example, the dataset may span from 0 to 36 hours, and the first time window may be 0 to 24 hours, 1-24 hours, 2-30 hours or 0-36 hours. For example, in one embodiment, a third order polynomial of the form shown in Equation 1 is fitted to the time-series data for each well:

y=A₀+A₁x+A₂x²+A₃x³ Equation 1

where x, the independent variable, is time; and y, the dependent variable, is the relative respiration index of the bacteria and indicated by a color change. The fitted coefficients A₀, A₁, A₂ and A₃, also known as regression coefficients, are summary parameters for the fit (or regression), and these summary parameters are then provided as the input features for the training dataset used to train the machine learning model. Additional summary parameters (or summary statistics) returned from the fitting method such as error term(s), a correlation coefficient, a coefficient of determination, ANOVA, etc may also be provided as part of the summary parameters. During training the input features are provided with a label indicating the host-phage response (e.g. 1=good/efficacious, 0=bad/ineffective) and used to train the machine learning model.

Fitting a function provides a way of summarising properties of the dataset to facilitate classification. Providing a series of raw images or even the complete time series dataset may lead to overfitting or provide too many parameters to enable efficiently classification. By fitting a function, properties of the dataset can be summarised, enabling more efficient and accurate classification. Thus, in the above embodiment, a third order polynomial was fitted. This was selected as it provides several fitted parameters (e.g. 4) which summarise the dataset for the machine learning model (achieving a data reduction), and a third order polynomial is able to capture both the curvature of the S shaped phage-host response data for ineffective phage, and as well as the approximately linear (non) growth curve of an efficacious phage as the higher order coefficients A₂ and A₃ will likely be close to zero. Such a function will also pick up partially effective phage. i.e. a curve between the extremes of uninhibited growth, and fully inhibited growth. However it will be understood that a range of other fitting functions could be used, including linear functions, quadratic, or higher order polynomials, as well as non-polynomial functions including log, exponential, power, trigonometric, B-splines, sigmoidal, non-linear functions, regression models, and combinations. Typically, the fitted function(s) will be parameterised by several parameters which can be provided as input to the machine learning model. The functions may be fitted using regression/curve fitting methods which attempt to minimise some parameter or loss function of the residuals with respect to the fitted function, including least squares based methods, and may use iterative, weighted and/or robust regression methods.

With reference to FIG. 2, it is also noted that the two extremes of uninhibited growth and fully inhibited growth are quite distinct functional forms—namely an approximately “S” shaped curve (i.e. sigmoidal) for uninhibited growth (or equivalently a ramped or slanted step function), compared to an approximately linear curve for inhibited growth. Thus in one embodiment, the fitted function may be selected to have a form that mimics one of the desired curves/case (phage efficacy), as it is noted that for the other case, the residuals will be large or heteroscedastic as the fitted function is not a good estimator/summary of the actual shape of the dataset. As such the residuals or errors will be large, or exhibit structure/not follow a zero mean normal distribution. Thus, in addition, or as an alternative, these residuals/errors, or summary parameters based on residuals/errors, may be provided as the parameters to train the model. The residuals/errors may be correlation coefficient r, coefficient of determination (R²), regression coefficients ε or an error matrix ε, or summary parameters/statistics of the residuals, such as the standard deviation, interquartile range, five number summary (min, lower quartile, median, upper quartile, max), several predefined quantiles (eg 10% & 90%) quantiles of the residual distribution. Further a goodness of fit test could be applied to the residuals and the output of the goodness of fit test used as the input to the machine learning model.

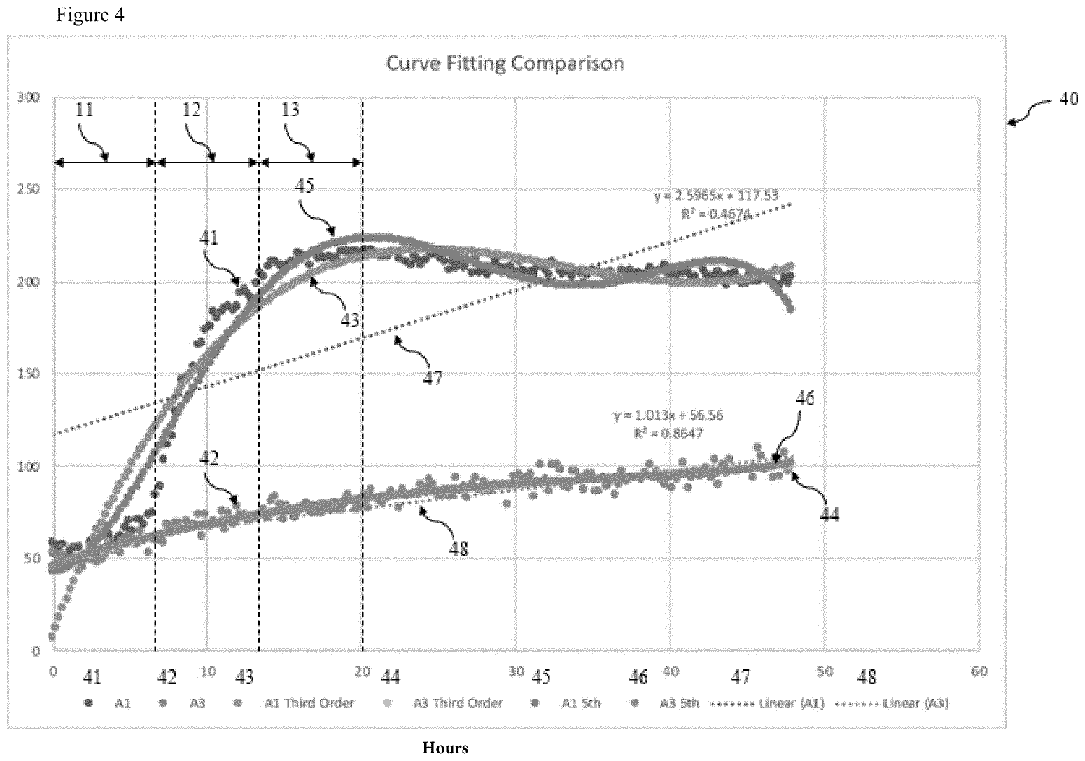

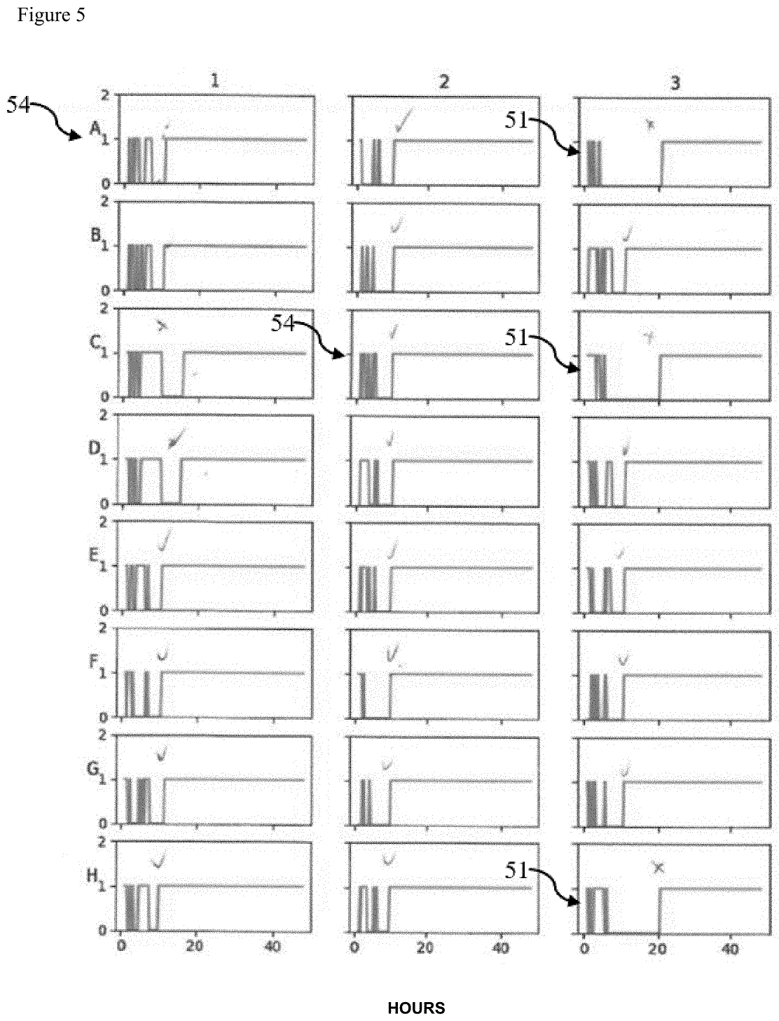

This is further illustrated in FIG. 4 which is a comparison of several curve fits on a first host-phage time series dataset 41 where the phage does not inhibit growth of the bacterial host (not efficacious/ineffective), and a second host-phage time series dataset 42 where the phage does inhibit growth of the bacterial host (efficacious/effective) according to an embodiment. A third order polynomial 43, a fifth order polynomial 45 and a linear fit 47 were each fitted to the first host-phage time series dataset 41. Similarly, a third order polynomial 44, a fifth order polynomial 46 and a linear fit 48 were each fitted to the second host-phage time series dataset 42. Table 1 lists the fitted model parameters.

TABLE 1
Fitted Model Parameters

| | ‘S’ Shape Curve | | ‘Linear’ Curve | |
|---|---|---|---|---|
| | 3^{rd} Order | 5^{th} Order | 3^{rd} Order | 5^{th} Order |
| A0 | 7.10994822 | 42.7370761 | 45.4850585 | 45.4211985 |
| A1 | 21.3065602 | 0.390008879 | 2.77824216 | 2.96280137 |
| A2 | −0.690963122 | 2.36230735 | −0.0600534319 | −0.101842681 |
| A3 | 0.00695951246 | −0.157364026 | 0.000537879196 | 0.00347344822 |
| A4 | | 0.00374796716 | | −0.0000798878794 |
| A5 | | −0.0000305126671 | | 0.000000738877893 |

It can be seen from FIG. 4 that the third and fifth order polynomials provided similar fitting performance, particularly in the case of the second dataset, where the curve was linear (Linear Curve) and thus the higher order coefficients (A2, A3, A4, A5) were effectively zero. Most noticeably the linear fit 47 on the first dataset was very poor due to growth induced curvature, generated a R² value of 0.467, whereas the linear fit models the second dataset well and has a R² value of 0.865. Thus, in addition to providing model estimates, providing a measure of the goodness of fit (e.g. R²) or some parameterisation of the residuals such as standard deviation, interquartile range of residuals provides additional information to the machine learning model to assist in separating host-phage response datasets. In some embodiments, several functions may be fitted over the same time period. For example, a linear function and a third order polynomial could both be fitted over the same 0-24 hour time period. In these embodiments the summary parameters comprise the fitted parameters (e.g. coefficients and/or errors) from both fitting functions.

Further, and as is apparent from FIGS. 2 and 4, the curves 10 show distinct phases—namely a lag phase 11, a (potential) growth phase 12, and stabilization phase 13. Thus, in one embodiment rather than fitting a single function over a first time period (e.g. from 0 to 20 hours), we perform multiple fits where each fit is over a time segment shorter than the first-time window. For example, in the above case we can perform a first fit over the lag phase, a second fit over the growth phase, and a third fit over the stabilisation phase. In this embodiment the first-time window is defined by the start of the earliest time segment and the end of the latest time segment. These fits may be piecewise or segmented fitting/regression in which the time segments are contiguous segments which span the time window (i.e. so each function is fitted over a different time window). For example a first function could be fitted over a first fitting time segment such as 0-7 hours, a second function could be fit over a second time segment such as 7-14 hours, and third function could be fit over a third time segment 14-20 hours to define a first time period of 0-20 hours. These time segments (or fitting time periods or time portions) may contiguously span the first time period (i.e. piecewise fits), or in some embodiments the time segments may non-contiguously span the first time window such that there is a time gap between the end of one time segment and the start of another time segment (e.g. 3-7 hours, 9-13 hours, 17-20 hours). Further the time segments may be partially overlapping time periods such that a portion of the end of one-time segment may overlap with a portion of the start of another time segment (e.g. 0-10 hours, 5-15 hours, 10-20 hours). In one embodiment the time segments could be fixed widths and the time segments are sliding time segments. The same type of fitting function (e.g. having the same functional form such as linear, third order polynomial, etc) may be fitted to each time segment (i.e. a single function is fitted to each distinct time segment), or multiple functions with different types or forms may be fitted to each time segment. For example, in one embodiment a piecewise linear fit is performed in at least 3 time segments, and the R² value for each time segment is provided as one of the input parameters to the machine learning model. A good phage is indicated by R² being close to 1 in each of the fitting time segments. In the case that different functional forms/types are fitted to each segment, these need not be restricted to being continuous over boundaries.

Thus, in view of the above we can generalise the fitting step (step 120 in FIG. 1A) as fitting, for each time series data set, at least one function over a first-time window. As discussed this could be a single function over a single time window, multiple functions over the same single time window, or fitting a plurality of functions each over a time segment wherein the first time window is defined by the start of the earliest time segment and the end of the latest time segment and each time segment is shorter than the first time window. The time segments may each be different time and may contiguously or non-contiguously span the first-time window. We then perform a summarizing step 130 in which the results of the fitting step are used to generate a set of summary parameters for each fit which is used to train the machine learning model (i.e. provided as the input to the machine learning model), and subsequently are used as the input to the trained model to classify a test host-phage response dataset. The summary parameters comprising one or more model coefficients/fitted parameters, goodness of fit, R², errors, residuals, or summary statistics of residuals.

Once a set of summary parameters for each dataset set is determined (or estimated), this can be used to create a training dataset (and a validation dataset) used as input to train a machine learning model. At step 140 we then proceed to train a machine learning model on a training dataset comprising the set of summary parameters for the fit to a time series dataset for a host-phage combination, and the associated label for the time series dataset. The input dataset may be formatted as a matrix, where each row represents a host-phage combination (or rather the time-series dataset of observations of the growth of the host in the presence of the phage in a well) and the columns represent the fitted coefficients. However, it will be understood that the dataset may be stored in other formats or representations across one or more storage devices including networked storage devices and/or databases. Labels can then be assigned to each row (e.g. added as an extra column) for training and validation assessment of the machine learning model.

In these embodiments, the machine learning algorithm is a supervised classification approach which once trained can be used to estimate (classify) the efficacy of a test phage against a test host bacterium from host-phage response datasets. A range of machine learning classifiers may be used, such as a Boosted Trees Classifier, Random Forest Classifier, Decision Tree Classifier, Support Vector Machine (SVM) Classifier, Logistic Classifier, etc. In some embodiments the classifier is a probabilistic classifier. That is rather than just issuing a binary classification (e.g. efficacious or not), the classifier outputs the class probability. Probabilistic classifiers include naïve Bayes, binomial regression models, discrete choice models, decision tree and boosting based classifiers.

Machine Learning training comprises separating the complete dataset into a first training dataset and a second validation dataset. The training dataset is preferably around 60-80% of the total dataset. This training dataset is used by machine learning models to create a classifier model to accurately identify efficacious phage. The second set is the Validation dataset, which is typically at least 10% of the dataset and more preferably 20-40%: This dataset is used to validate the accuracy of the model created using the training dataset. Data may be randomly allocated to the training dataset and validation datasets. In some embodiments, checks may be performed on the training dataset and validation datasets to ensure a similar proportion of good/bad phage are present in each.

In some embodiment a plurality of training-validation cycles are performed (cross validation). In each train-validate cycle the dataset is randomly allocated to the training and validation datasets and used to train a model. This is repeated many times, and the best model selected or multiple good performing models from different cycles may be identified, and the results combined using an ensemble voting approach. For example, each model could vote on whether it predicts the phage if efficacious or not and a majority rule used to output the classification. Such methods can also provide coarse confidence estimates, for example based on the size of majority.

In some embodiments where cross-validation is used the dataset may be allocated to three datasets, namely a training dataset, a validation dataset, and a holdout or test dataset. The third holdout or test dataset is typically around 10-20% of the total dataset and is not used for training the machine learning classifier or the cross validation. This holdout dataset provides an unbiased estimate of the accuracy of the machine learning classifier model.

Once the machine learning model is trained, we then export or save the machine learning model in an electronic format at step 150, for subsequent use by a computing system (the same or a different computing system), to estimate the efficacy of a test phage in inhibiting growth of a test bacteria using a host phage response time series dataset obtained using the test phage and test bacteria. The model can be exported or saved to an electronic model file using an appropriate function of the machine learning code/API for loading onto another computer device which is configured to execute the model to classify new host-phage response data. In some embodiments the machine learning model is saved for later use on the same computing device used to train the machine learning model. The electronic model file may be an electronic file generated by the machine learning code/library with a defined format which can be exported and then read back in (reloaded) using standard functions supplied as part of the machine learning code/API (e.g. exportModel( ) and loadModel ( )). The file format may be binary format, including a machine readable format, or a text format, and may be a serialised representation. The electronic file may be sent to another computing system or saved to a storage location, including a network storage location, using JSON, YAML or similar data transfer protocols. In some embodiments additional model metadata may be exported/saved and sent along with the model parameters, such as model accuracy, training dataset description, etc., that may further characterise the model, or otherwise assist in constructing another model on another computing device/server.

At step 160 the machine learning model is then used, by a computing system or apparatus, to estimate the efficacy of a test phage in inhibiting growth of a test bacteria using a host phage response time series dataset obtained using the test phage and test bacteria. This is further illustrated in flowchart FIG. 1B which is a flowchart of a method for interpreting host phage response data 200. This can be executed on the same computer system or apparatus or another computer system or apparatus using the trained machine learning model.

At step 210, we load the trained machine learning model configured to classify host response dataset into a computing system. This may comprise receiving the electronic file exported in step 150 which describes the trained machine learning model and reading (by the computing system) the electronic file to reconstruct the trained machine learning model in the memory for execution by the processor(s). To be clear, this does not require the training data and need only describe or characterize the configuration of the classifier which was learned from the training data. At step 220 we receive a host response dataset for a test phage. This may be uploaded to the computing system via a webportal, or sent as an electronic file by a computing apparatus associated with the apparatus which generated the host response dataset, or the computing apparatus associated with the apparatus which generated the host response dataset may store the host response dataset as an electronic file in the storage location (such as network storage), and the computing system may periodically poll the storage location for newly received files in the storage location. As in the case of the training datasets, the dataset comprises a time series dataset where each data point in the time series dataset comprises a measurement of a parameter indicative of the growth of a host bacteria in the presence of a test phage at a specific time. At step 230 we then fit at least one function over a first time window and then at step 240 we generate a set of summary parameters for the fit (e.g. model parameters and/or error/residual estimates). Steps 230 and 240 are equivalent to steps 120 and 130 so that the input data to the trained machine learning model has been generated in the same way as the training data. Note that as we are passing a set of summary parameters, the time window over which the fit (or fits) is performed need not be identical to the time window used for training. However it is preferable that the time window is the same or similar, or at least sufficient for the fit to obtain reliable estimators of fitted parameters. Similarly, the same fitting process used during training the machine learning model should be used to generate equivalent summary parameters for classification by the machine learning model. For example whether we fit a single function over the first time window, multiple functions over the first time window, or a plurality of functions each fitted over a time period which is a portion of the first time window, is determined based on how the machine learning model was trained so that equivalent summary parameters can be generated. At step 250 we then obtain an estimate of the efficacy of the test phage in inhibiting growth of the host/test bacteria by providing the set of summary parameters to the machine learning model. i.e. the trained machine learning model classifies the input dataset. At step 260 we then report the estimate of the efficacy of the test phage. This report may be a binary output, such as the phage is efficacious or not (i.e. ineffective). In some embodiments the machine learning model may also output a confidence estimate of the classification. The report may be an electronic record, such as PDF file, or it may be an electronic report provided via a user interface of the computing system. For example the web interface used to upload the host response dataset may also be used to publish the report, for example using an automated report generator module (eg Microsoft reporting services) which generates a report using a stored template which when executed incorporates the estimate of the efficacy. Further the system may be configured to allow users to upload multiple host response datasets and report all the results in a single report.

Table 2 shows the validation results from various machine learning models tested on a data set comprising 1000 rows. This dataset was split into a training set comprising 80% of the data and a test set comprising the remaining 20% of the data.

TABLE 2
Machine Learning Model
Validation Results

| | Validation |
|---|---|
| ML Model | Accuracy |
| Boosted Trees Classifier | 0.94444 |
| Random Forest Classifier | 0.96296 |
| Decision Tree Classifier | 0.96296 |
| SVM Classifier | 0.92593 |
| Logistic Classifier | 0.96296 |

The Random Forest Classifier, Decision Tree Classifier, and Logistic Classifier were the best performing classifiers on this data set. However, the performance of the Boosted Trees Classifier and even the SVM Classifier were only slightly lower than these three models. Further given the accuracy would be expected to vary from test run to test run, this indicates that any of the above machine learning models are likely to be acceptable. In one embodiment the Machine Learning model is either a Random Forest Classifier, a Decision Tree Classifier, or a Logistic Classifier.

From FIG. 4 it can be seen that after the lag phase (i.e. around 6-7 hours), the two curves start to diverge. Thus, to examine how quickly the model can distinguish poorly performing phage, an in silico experiment was performed to see how quickly the machine learning model could reliably classify a test host-phage response dataset. In this experiment, a machine learning model was fitted to the complete dataset for each host-phage response, and then a series of test fits was then performed on the dataset where each fit was performed over a progressively increasing time window, using 15-minute intervals. That is a test fit was performed over a time window (0, t) where t was incremented by 15 minutes for each subsequent test fit and the fitted parameters were then provided to the trained machine learning model. As noted above, the trained machine learning model only requires a set of summary parameters, and the time window of the fitted dataset need not be identical to that used to train the model.

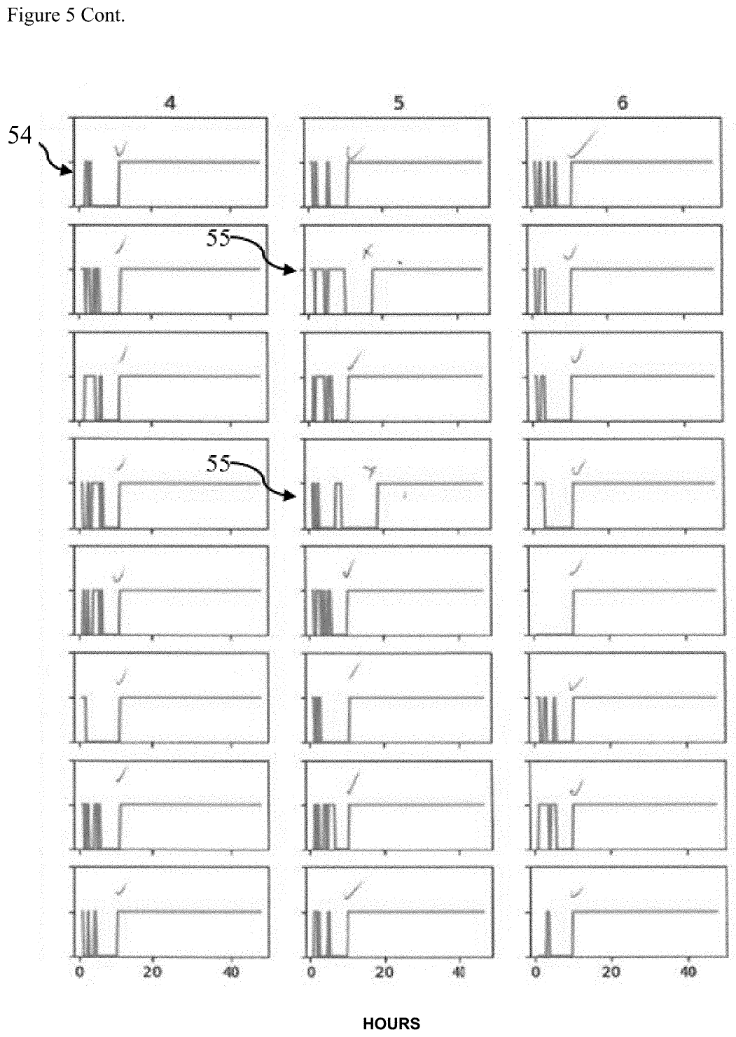

FIG. 5 is a set of 48 plots showing the time taken for a machine learning model to correctly classify the efficacy of a host-phage time series dataset according to an embodiment. Each of the plots shows whether the classification on the test fit agrees with the classification obtained from the machine learning model on the complete dataset, where “1” signifies agreement, and “0” signifies disagreement at each of the 15-minute intervals. Each plot thus shows how long it takes for the machine learning algorithm to generate the correct/stable estimate.

Not surprisingly, the plots fluctuate significantly in the first few hours but tend to settle on the correct estimate between 10-20 hours. Notably A3, C3 and H3 are cases where the phage are effective at inhibiting growth, and these each take around 20 hours (timepoint 51) for the Machine learning model to make a reliable estimate. This is contrasted with A1, A4 and C2 where the phage is not effective, and these achieve stable estimates after 10 hours (time point 54). However, some cells with ineffective phage such as B5 and D5 take longer to stabilize (time point 55).

These results suggest that the machine learning model is reasonably accurate at quickly predicting poor phage after 10 hours but that it takes longer for effective phage to be identified—in this case around 20 hours). This suggests that the time period should span 20 hours, although tests could be performed after 10 hours to select out clearly ineffective phage. The minimum desirable time period will however depend to some extent on the fitting function used, time window of which fits are performed (e.g. single or piecewise fits), and growth media for the wells used for the host-phage response tests.

In one embodiment, the fitting step could be performed repeatedly during the course of the host phage experiment. That is, as the experiment progresses, and further images and data becomes available, the dataset is updated with the additional data points (i.e. the additional times) and the fitting function is refitted and classified on the updated dataset. This is equivalent to progressively increasing the time window with each new fit. In another embodiment the width of the fitting time window could be fixed such that the fitting process is effectively using a sliding time window as further data becomes available. In these embodiments, a probabilistic classifier may be used to output the classification probability. Alternatively, a classification expectancy could be estimated with each new time point/fit. The classification expectancy is an estimate of the probability (or likelihood) that the classification result being correct conditional on the current state determined using the distributions of historical data which contain a point matching the current state at the current time. That is, given set of parameters at a given time in the assay, a number could be produced that is measure of the confidence of the classification outcome (i.e. is the current classification result the expected result) for a given phage. For example, new data could be obtained every 15 minutes, and the classifiers decision could be saved for each time point. To obtain the classification expectancy at each point we extract the subset of the historical dataset that had a matching current state. In a first embodiment this could be the dataset with the same classification outcome at the current time point. Having obtained this subset we then determine the percentage of the subset where the current estimate of the classification result was the same as the final classification result (e.g. the classification after completion of the assay) and we return that percentage (or a number based on that percentage). As time progresses this is expected to stabilise on the final value. That is, for an assay performed over 24 hours, we may get a classification result at 4 hours with a probability of 50% (i.e. unstable estimate). By 12 hours the probability may be 75% (likely to be accurate), and by 20 hours it may be 99% (highly likely to be accurate). In another embodiment, the dataset could be the dataset with the same classification outcome at the current time point and with growth measure (ie a time series value) within some predefined range of the observed growth measure (ie the time series value) at the current time. This could be achieved by partitioning the growth values (y axis values in FIG. 2) into a set of intervals or bins (e.g. 0 to 0.1, 0.1 to 0.2, 0.3 to 0.4, etc). We then identify which interval/bin the observed growth measure falls within and select the subset of historical data that had observed growth measures in the same interval/bin at the same time with the same current classification result. Having obtained this subset, we then determine the percentage of the subset where the current classification result was the same as the final classification result (i.e. is the current classification result the expected classification result). In an alternative embodiment, the dataset could be the dataset with the growth measure within some range of the observed growth measure at the current time as described above (i.e. selection of the dataset ignores the current classification result). We then return the percentage of final classification result for this subset which had a final classification result that matches the current classification result. The classification expectancy can thus provide an early measure of the confidence or stability of the current classification result by leverages the longer time series (and outcomes) available in a historical dataset.

The above embodiments can be used to identify one or more efficacious phage for a host bacterium. For example, in FIG. 5, 3 efficacious phages (A3, C3 and H3) were identified. Where multiple phages are tested against the same host bacteria, a therapeutic phage formulation of the most effective phage(s) can be generated for treatment. Selection of which phage, or phages, to include may be obtained using a measure of diversity of the efficacious phage. In one embodiment the measure of diversity is indicative of a different mechanism of action between the phage. This measure of diversity could be estimated by sequencing the phage and using bioinformatics methods or datasets to estimate functional effects/associations and these could be used to assign one or more mechanism of actions labels (these could be selected from a controlled ontology such as the GeneOntology database, or databases of biological networks). Phage combinations can thus be selected based on those with different mechanisms of actions, or where phage are assigned a set of multiple possible mechanisms of action, phage could be selected based on the two phage with most dissimilar sets (i.e. minimum overlap of possible mechanisms of action). Overlapping methods of actions could be defined based on sharing a biological network or pathway, or a GeneOntology (GO) term (or downstream of a GO term), or a GO-CAM model. For example, each pair of phage could be assigned a score based on the number of mechanism of actions not shared by both lists. The largest score would indicate the most diverse (non overlapping) list. In another example, the score could be a weighted score. For example, the previous score could be divided by sum of the two list sizes to weight for list size. Other weighting or scoring functions could be used, such as applying a weighting that takes into account the evidence for a mechanism of action associated with a sequence. Other methods of assessing diversity of possible mechanisms of action could also be used based on bioinformatics data mining or biological network/pathway analysis. This approach provides robustness against a bacterium adapting to the mechanism of action of a single phage, as if the second phage has a different mechanism of action then it is likely to remain effective.

Embodiments described herein thus advantageously provide automated methods for analysing/interpreting host phage response data. By using an approach where one or more functions are fit and generating summary parameters as input for training of a machine learning model, a machine learning model can be efficiently trained as a classifier. The method of using the summary data is largely independent of the data size and sampling frequency when deployed, i.e. if the data is sampled every minute or every 15 minutes the training and subsequent deployment still reduces to the summary parameters calculated. The approach can be used to identify phage for including in phage formations for treating patients with bacterial infections, and in particular Multiple Drug Resistant Infections. The methods can also be used to identify phage that can be used to clean up bacterial contaminated areas, such as for cleaning up an industrial site. These phage formulations may include two or more phage with different mechanisms of action as described above.

Variations on the above methods can also be performed. In one embodiment, the historical dataset is used to improve classification when performed during the assay (i.e. at some time point before the full assay time period). In this embodiment, a fit (or multiple fits) is performed over the current time period (e.g. 0 to 6 hours). Then fit results over the same time period is obtained for each host-phage profile in a historical dataset is obtained, and a subset of the historical dataset is selected based on having fit results similar to the fit results for the current host-phage combination (over the current time period). That is, we identify the subset of the historical dataset with a similar phage-host curve to the observed phage-host curve up to this point in time (or over some time range to this point in time). Determining similar phage-host curves could be performed using correlation measures (e.g. a cross correlation or similar similarity measure). We then provide additional data from the historical dataset as further inputs to the classifier (beyond just the fit values). In one embodiment this might be percentage of this subset of the historical dataset which were ultimately efficacious.

In one embodiment, a deep learning method may be used to generate a model where large amounts of host-phage response training data are available. In deep learning methods a neural network, which typically comprises many layers of convolutional neural nets with a classification layer, is trained by optimizing the parameters or weights of the model to minimize a task-dependent ‘loss function’. For example, if we consider a Binary Host-Phage Response Classification problem, that is, separating a set of host-phage response time series into exactly two categories, the fitted function parameters are run through the model which computes a binary output label e.g. 0 or 1—to represent the two categories of interest. The predicted output is then compared against a ground truth label, and a loss (or error) is calculated. In the binary classification example, a Binary Cross-Entropy loss function is the most commonly used loss function. Using the loss value obtained from this function, we can compute the error gradients with respect to the input for each layer in the network. This process is known as back-propagation. Intuitively, these gradients inform the network how to modify (or optimize) the weights to obtain a more accurate prediction for each of the images.

In practice however, it may be difficult, inadvisable or even impossible to compute the network update in a single iteration or ‘epoch’ of training. Often, this is due to the networks requiring a large amount of data and containing a large number of parameters that can be modified. To solve this, often, mini-batches of data are used in place of the full set. Each of these batches is drawn at random from the dataset, and a large enough batch size is chosen to approximate the statistics for the entire dataset. The optimization then is applied over the mini-batches until a stopping condition is met (i.e. until convergence, or satisfactory results according to a pre-defined metric are achieved). This process is known as Stochastic Gradient Decent (SGD) and is the standard process of optimizing neural networks. Usually, the optimizer is run for hundreds of thousands to millions of iterations. Furthermore, Neural network optimization is non-convex, and there are often many local minima in the parameter space defined by the loss function. Intuitively, this means that due to the complex interactions among the weights in the network and the data, there are many almost-equally valid combinations of weights that result in almost-identical outputs. Deep Learning models, or neural network architectures that contain many layers of convolutional neural nets are typically trained using Graphics Processing Units (GPUs). GPUs are extremely efficient at computing Linear Algebra compared with Central Processing Units (CPUs).

Like machine learning training, training a neural net comprises performing a plurality of training-validation cycles. In each train-validate cycle each randomization of the total useable dataset is split into at 3 datasets. As before the first data set is the training dataset and preferably is around 70-80% of the total dataset: This dataset is used to create a classifier model to accurately identify efficacious phage based on the labelled training data. The second set is the validation dataset, which is typically at least 10% of the dataset. This dataset is used to validate or test the accuracy of the model created using the training dataset. Even though this dataset is are independent of the training dataset used to create the model, the validation dataset still has a small positive bias in accuracy because it is used to monitor and optimize the progress of the model training. Hence, training tends to be targeted towards models that maximize the accuracy of this particular validation dataset, which may not necessarily be the best model when applied more generally to other datasets. Thus, it is often preferred (but not necessary) to have a third dataset known as the blind validation dataset which is typically around 10-20% of the dataset. This validation occurs at the end of the modelling and validation process, when a final model has been created and selected, and is used conduct a final unbiased accuracy assessment of the final model and address any positive bias with the validation dataset. The accuracy of the validation dataset will likely be higher than the blind validation dataset for the reasons discussed above, however the results of the blind validation dataset are a more reliable measure of the accuracy of the model.

Machine Learning models are trained using a plurality of Train-Validate cycles on a dataset. For ease of understanding, the dataset can be formatted as a matrix, where each row represents a host-phage experiment (time-series) and the columns represent the fitted coefficients. However, it will be understood that the dataset may be stored in other formats or representations across one or more storage devices including networked storage devices. The Train-Validate cycle follows the following framework.

The training data are split into batches. The number of rows (time series) in each batch is a free model parameter but controls how fast and how stably the algorithm learns. After each batch, the weights of the network are adjusted, and the running total accuracy so far is assessed. When all rows have been assessed we say one epoch has been carried out. The training set is then re-randomized, and the training starts again from the top, for the next epoch. During training a number of epochs may be run, with the number depending on the size of the dataset, the complexity of the dataset and the complexity of the model being trained. In some embodiments the number of epochs may be anywhere from 100 to 1000 or more. After each epoch, the model is run on the validation set, without any training taking place, to provide a sense of the progress in how accurate the model is. This may be used to guide the user or system on whether more epochs should be run, or if more epochs will result in overtraining. The validation set guides the choice of the overall model parameters (hyperparameters) and is therefore not a truly blind set. Once the model is trained, the blind validation dataset is used to assess final accuracy.

In deep learning, a range of free parameters is used to optimize the model training on the validation set. One of the key parameters is the learning rate, which determines by how much the underlying neuron weights are adjusted after each batch. Typically, when training a model, we try and avoid overtraining, or overfitting the data. This happens when the model contains too many parameters to fit, and essentially ‘memorizes’ the data, trading generalizability for accuracy on the training or validation sets. The likelihood of overtraining can be ameliorated through a variety of tactics, including slowed or decaying learning rates (e.g. halve the learning rate every n epochs), tensor initialization, pre-training (using a previous trained model as the starting point), and the addition of noise, such as Dropout layers, or Batch Normalization, which force the model to generalize more truly. Dropout regularization effectively simplifies the network by introducing a random chance to set all incoming weights zero within a rectifier's receptive range. By introducing noise, it effectively ensures the remaining rectifiers are correctly fitting to the representation of the data, without relying on over-specialization. This allows the neural net to generalize more effectively and become less sensitive to specific values of network weights. Similarly, batch normalization can allow faster learning and generalization by shifting the input weights to zero mean and unit variance as a precursor to the rectification stage.

In performing deep learning, the methodology for altering the neuron weights to achieve an acceptable classification includes the need to specify an optimization protocol. That is, for a given definition of ‘accuracy’ or ‘loss’ (discussed below) exactly how much the weights should be adjusted, and how the value of the learning rate should be used, has a number of techniques that need to be specified. Suitable optimisation techniques include Stochastic Gradient Descent (SGD) with momentum (and/or Nesterov accelerated gradients), Adaptive Gradient with Delta (Adadelta), Adaptive Moment Estimation (Adam), Root-Mean-Square Propagation (RMSProp), and Limited-Memory Broyden-Fletcher-Goldfarb-Shanno (L-MBFGS) Algorithm. In addition to these methods, it is also possible to include non-uniform learning rates. That is, the learning rate of the convolution layers can be specified to be much larger or smaller than the learning rate of the classifier. This is useful in the case of pre-trained models, where changes to the filters underneath the classifier should be kept more ‘frozen’, and the classifier be retrained, so that the pre-training is not undone by additional retraining.

While the optimizer specifies how to update the weights given a specific loss or accuracy measure, in some embodiments the loss function is modified to incorporate distribution effects. These may include cross-entropy loss, inference distribution or a custom loss function.

Cross Entropy Loss is a commonly used loss function, which has a tendency to outperform simple mean-squared-of-difference between the ground truth and the predicted value. If the result of the network is passed through a Softmax layer, then the distribution of the cross entropy results in better accuracy. This is because is naturally maximizes the likelihood of classifying the input data correctly, by not weighting distant outliers too heavily. For an input array, batch, representing a batch of host-phage time series, and class representing efficacy (i.e. is the phage good or poor at inhibiting bacterial growth), the cross entropy loss is defined as:

$$loss ( batch , class ) = - log ( \frac{exp ( batch ) [ class ] )}{{\sum }_{j} exp ( x [ j ] )} ) \qquad ( 2 )$$

If the data contains a class bias, that is, more poor than good phage examples (or vice-versa), the loss function should be weighted proportionally so that misclassifying an element of the less numerous class is penalized more heavily. This is achieved by pre-multiplying the right hand side of Equation (2) with the factor: weight[class]=1/N[class] where N[class] is the total number of datasets for each class. It is also possible to manually bias the weight towards the good phage in order to reduce the number of false negatives compared to false positives, if necessary.

In some embodiments an Inference Distribution may be used. While it is important to seek a high level of accuracy in classifying phage, it is also important to seek a high level of transferability in the model. That is, it is often beneficial to understand the distribution of the scores, and that while seeking a high accuracy is an important goal, the separation of the efficacious (good) and non-efficacious (poor) phage confidently with a margin of certainty is an indicator that the model will generalize well to a holdout test set. Since the accuracy on the test set can be used benchmarking, such as the comparing the accuracy of the skilled analyst classifying the same phage-host graph, ensuring generalizability should also be incorporated into the batch-by-batch assessment of the success of the model, each epoch.

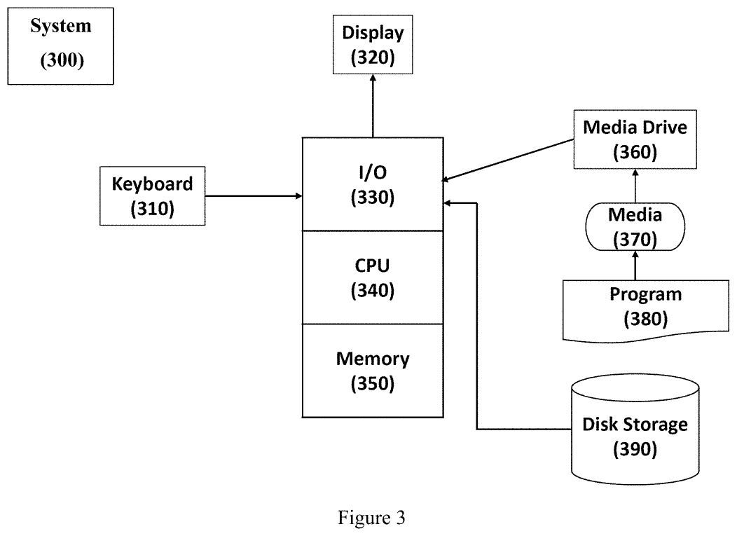

FIG. 3 depicts an exemplary computing system configured to perform any one of the computer implemented methods described herein. The computing system may comprises one or more processors operatively connected to one or more memories which store instructions to configure the processor to perform embodiments of the method. In this context, the computing system may include, for example, one or more processors, memories, storage, and input/output devices (e.g., monitor, keyboard, disk drive, network interface, Internet connection, etc.). However, the computing system may include circuitry or other specialized hardware for carrying out some or all aspects of the processes. The computing system may be a computing apparatus such as an all-in-one computer, desktop computer, laptop, tablet or mobile computing apparatus and any associated peripheral devices. The computer system may be a distributed system including server based systems and cloud-based computing systems. In some operational settings, the computing system may be configured as a system that includes one or more units, each of which is configured to carry out some aspects of the processes either in software, hardware, or some combination thereof. For example the user interface may be provided on a desktop computer or tablet computer, whilst the training of the machine learning model and execution of a trained machine learning model may be performed on a server based system including cloud based server systems, and the user interface is be configured to communicate with such servers. The user interface may be provided as a web portal, allowing a user on one computer to upload datasets which may be processed on a remote computing apparatus or system (eg server or cloud system) and which provides the results (ie the report) back to the user, or to other users on other computing apparatus

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Specifically, FIG. 3 depicts computing system (300) with a number of components that may be used to perform the processes described herein. For example, an input/output (“I/O”) interface 330, one or more central processing units (“CPU”) (340), and a memory section (350). The I/O interface (330) is connected to input and output devices such as a display (320), a keyboard (310), a disk storage unit (390), and a media drive unit (360). The media drive unit (360) can read/write a computer-readable medium (370), which can contain programs (380) and/or data. The I/O interface may comprise a network interface and/or communications module for communicating with an equivalent communications module in another device using a predefined communications protocol (e.g. Bluetooth, Zigbee, IEEE 802.15, IEEE 802.11, TCP/IP, UDP, etc). This may be a single computing apparatus, or a distributed computing apparatus or distributed computing system including cloud based computing systems.

In one embodiment the machine learning model was generated using Turi Create (apple.github.io/turicreate) which is an python based machine learning library developed by Apple (and earlier Turi) for building AI/Machine learning based application. However, in other embodiments similar machine learning libraries/packages such as SciKit-Learn, Tensorflow, and PyTorch, may be used. These typically implement a plurality of different classifiers such as a Boosted Trees Classifier, Random Forest Classifier, Decision Tree Classifier, Support Vector Machine (SVM) Classifier, Logistic Classifier, etc. These can each be tested, and the best performing classifier selected. A computer program may be written, for example, in a general-purpose programming language (e.g., Pascal, C, C++, Java, Python, JSON, etc.) or some specialized application-specific language to provide a user interface, call the machine learning library, and export results.

A non-transitory computer-program product or storage medium comprising computer-executable instructions for carrying out any of the methods described herein can also be generated. A non-transitory computer-readable medium can be used to store (e.g., tangibly embody) one or more computer programs for performing any one of the above-described processes by means of a computer. Further provided is a computer system comprising one or more processors, memory, and one or more programs, wherein the one or more programs are stored in the memory and configured to be executed by the one or more processors, the one or more programs including instructions for carrying out any of the methods described herein.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A computer implemented method for training a machine learning model for interpreting host phage response data, the method comprising:
receiving or uploading, by a computing system, a host phage response dataset and labels, wherein the host phage response dataset comprises a time series dataset for each of a plurality of host-phage combinations in which a host bacteria is grown in the presence of a phage, and each data point in the time series dataset associated with a host-phage combination comprises a measurement of a parameter indicative of the growth of the respective host bacteria in the presence of the respective phage at a specific time, and each time series dataset has an associated label indicating an efficacy of the phage in inhibiting growth of the host bacteria;
fitting, for each time series dataset, at least one function over a first time window;
generating a set of summary parameters for each fit, the summary parameters comprising one or more model coefficients, goodness of fit, R², errors, residuals, or summary statistics of residuals; and
training a machine learning model on a training dataset comprising the set of summary parameters for each fit to one of the time series datasets, and the associated label for the fitted time series dataset;
exporting or saving the machine learning model in an electronic format for subsequent use to estimate an efficacy of a test phage in inhibiting growth of a test bacteria using a host phage response time series dataset obtained using the test phage and test bacteria.

2. The computer implemented method as claimed in claim 1, wherein fitting, for each time series dataset, at least one function over a first time window comprises fitting a single function over the first time window.

3. The computer implemented method as claimed in claim 1, wherein fitting, for each time series data set, at least one function over a first time window comprises fitting at least two functions over the first time window, wherein each of the functions have a different functional form.

4. The computer implemented method as claimed in claim 1, wherein fitting, for each time series data set, at least one function over a first time window comprises performing a plurality of fits, wherein each fit comprises fitting a function over a time segment wherein the first time window is defined by a start of the earliest time segment and the end of a latest time segment and each time segment is shorter than the first time window.

5. The computer implemented method as claimed in claim 4, wherein the time segments are non-contiguous time segments.

6. The computer implemented method as claimed in claim 4, wherein a number of time segments is at least three.

7. The computer implemented method as claimed in claim 1, wherein an end of the first time period is 24 hours or less.

8. The computer implemented method as claimed in claim 1, wherein the at least one function is one or more of a linear function or a polynomial functional.

9. The computer implemented method as claimed in claim 1, wherein the machine learning model is a binary classifier which generates a binary outcome indicating whether a test phage is efficacious in inhibiting growth of a test bacteria or not.

10. The computer implemented method as claimed in claim 1, wherein the machine learning model is a probabilistic classifier which estimates a probability that a test phage is efficacious in inhibiting growth of a test bacteria.

11. A computer implemented method for interpreting host phage response data, the method comprising:
loading, by a computing system, a trained machine learning model stored in an electronic format and configured to classify host response dataset;
receiving and/or uploading a host response dataset for a test phage, wherein the host response dataset comprises a time series dataset where each data point in the time series dataset comprises a measurement of a parameter indicative of the growth of a host bacteria in the presence of the test phage at a specific time;
fitting at least one function over a first time window;
generating a set of summary parameters for the fitting;
obtaining an estimate of an efficacy of the test phage in inhibiting growth of the host bacteria by providing the set of summary parameters to the trained machine learning model; and
reporting the estimate of the efficacy of the test phage.

12. The computer implemented method as claimed in claim 11, further comprising receiving an updated host response dataset comprising additional data points and repeating the fitting, generating, obtaining and reporting steps, wherein reporting the estimate includes an estimate of a probability that the test phage is efficacious.

13. The computer implemented method as claimed in claim 11, further comprising determining a classification expectancy and reporting the estimate of the efficacy of the test phage further comprises reporting the classification expectancy, wherein determining the classification expectancy comprises:
selecting a subset of a historical host response dataset based on host-phage combinations with, at the end of a first time window, a state matching a current state of the host response dataset for the test phage, wherein the historical host response dataset comprises a time series dataset for each of a plurality of host-phage combinations, and each data point in the time series dataset associated with a host-phage combination comprises a measurement of a parameter indicative of the growth of the respective bacteria in the presence of the respective phage at a specific time, and each time series dataset has an associated estimate of an efficacy of the phage in inhibiting growth over an assay time period;
determining a classification expectancy by determining a percentage of the subset of the historical host response dataset with an estimate of the efficacy of the phage in inhibiting growth over the assay time period that matches the estimate of the efficacy of the test phage.

14. The computer implemented method as claimed in claim 13, wherein determining the state matching a current state of the host response dataset for the test phage is determined based on a host-phage combination having a classification output at a time matching the end of the first time window matching the estimate of the efficacy of the test phage.

15. The computer implemented method as claimed in claim 13, wherein determining the state matching a current state of the host response dataset for the test phage is determined based on a host-phage combination having a classification output at a time matching the end of the first time window matching the estimate of the efficacy of the test phage and a time-series value at the end of the first time window within a predetermined range of a time-series value of the test phage at the end of the first time window.

16. The computer implemented method as claimed in claim 13, further comprising receiving an updated host response dataset comprising additional data points and repeating the fitting, generating, obtaining and reporting steps, wherein reporting the estimate includes an updated estimate of the classification expectancy.

17. The computer implemented method as claimed in claim 11, wherein the method is repeated for a plurality of host response datasets, and the method further comprises:
obtaining a set of at least two test phage estimated as efficacious against a test bacteria;
obtaining estimates of one or more mechanisms of action for each test phage in the set;
obtaining a measure of diversity for each pair of test phage in the set based on the estimated mechanisms of action for each test phage;
selecting at least two phage for use in a therapeutic phage formulation based on the obtained measures of diversity.

18. The method of claim 17, wherein the mechanism of action is measured by sequencing the test phage.

19. A non-transitory, computer program product comprising computer executable instructions for training a machine learning model for interpreting host phage response data, the instructions executable by a computer to:
receive a host phage response dataset and labels, wherein the host phage response dataset comprises a time series dataset for each of a plurality of host-phage combinations in which a host bacteria is grown in the presence of a phage, and each data point in the time series dataset associated with a host-phage combination comprises a measurement of a parameter indicative of the growth of the respective bacteria in the presence of the respective phage at a specific time, and each time series dataset has an associated label indicating the efficacy of the phage in inhibiting growth of the host bacteria;
fit, for each time series data set, at least one function over a first time window;
generate a set of summary parameters for each fit, the summary parameters comprising one or more model coefficients, goodness of fit, R², errors, residuals, or summary statistics of residuals; and
train a machine learning model on a training dataset comprising the set of summary parameters for each fit to one of the time series datasets, and the associated label for the fitted time series dataset;
export the machine learning model in an electronic format.

20. A non-transitory, computer program product comprising computer executable instructions for interpreting host phage response data, the instructions executable by a computer to:
load a trained machine learning model configured to classify host response dataset;
receive a host response dataset for a test phage, wherein the host response dataset comprises a time series dataset where each data point in the time series dataset comprises a measurement of a parameter indicative of the growth of a host bacteria in the presence of the test phage at a specific time
fit at least one function over a first time window;
generate a set of summary parameters for the fitting;
obtain an estimate of an efficacy of the test phage in inhibiting growth of the host bacteria by providing the set of summary parameters to the trained machine learning model; and
report the estimate of the efficacy of the test phage.

21. A computing apparatus comprising:
at least one memory, and
at least one processor wherein the memory comprises instructions to configure the processor to:
receive a host phage response dataset and labels, wherein the host phage response dataset comprises a time series dataset for each of a plurality of host-phage combinations in which a host bacteria is grown in the presence of a phage, and each data point in the time series dataset associated with a host-phage combination comprises a measurement of a parameter indicative of the growth of the respective host bacteria in the presence of the respective phage at a specific time, and each time series dataset has an associated label indicating the efficacy of the phage in inhibiting growth of the host bacteria;
fit, for each time series data set, at least one function over a first time window;
generate a set of summary parameters for each fit, the summary parameters comprising one or more model coefficients, goodness of fit, R², errors, residuals, or summary statistics of residuals; and
train a machine learning model on a training dataset comprising the set of summary parameters for each fit to one of the time series datasets, and the associated label for the fitted time series dataset;
export or save the machine learning model in an electronic format, wherein in use, the trained machine learning model is used to estimate the efficacy of a test phage in inhibiting growth of a test bacteria using a host phage response time series dataset obtained using the test phage and test bacteria.

22. A computing apparatus comprising:
at least one memory, and
at least one processor wherein the memory comprises instructions to configure the processor to:
load a trained machine learning model configured to classify a host response dataset;
receive a host response dataset for a test phage, wherein the host response dataset comprises a time series dataset where each data point in the time series dataset comprises a measurement of a parameter indicative of the growth of a host bacteria in the presence of the test phage at a specific time;
fit at least one function over a first time window;
generate a set of summary parameters for the fit;
obtain an estimate of an efficacy of the test phage in inhibiting growth of the host bacteria by providing the set of summary parameters to the trained machine learning model; and
report the estimate of the efficacy of the test phage.

23. A therapeutic phage formulation comprising at least two phage, wherein the at least two phage were selected by.
obtaining a set of at least two test phage estimated as efficacious against a test bacteria through using a trained machine learning model configured to interpret host phage response data for a plurality of host-phage combinations in which a host bacteria is grown in the presence of a phage;
obtaining estimates of one or more mechanisms of action for each test phage in the set;
obtaining a measure of diversity for each pair of test phage in the set based on the estimated mechanisms of action for each test phage;
selecting at least two phage for use in the therapeutic phage formulation based on the obtained measures of diversity.

24. The therapeutic phage formulation of claim 23, wherein the mechanism of action is measured by sequencing the test phage.
